(19) United States
(12) Patent Application Publication
(10) Pub. No.: US 2015/0098931 A1
(43) Pub. Date: Apr. 9, 2015

(54) Cooperative Medication Combination Systems

(71) Applicant: Kevin Ray Pickering, Peoria, AZ (US)

(72) Inventor: Kevin Ray Pickering, Peoria, AZ (US)

(73) Assignee: Kevin Ray Pickering, Peoria, AZ (US)

(21) Appl. No.: 14/048,694

(22) Filed: Oct. 8, 2013

(51) Int. Cl. A61K 45/06

(52) U.S. Cl. CPC A61K 45/06
USPC 424/94.1

(57) ABSTRACT

This invention aims to capture and teach the high-level concept of combining doses of medications in unconventionally substandard amounts, for the treatment of medical. pathologies. By combining multiple medications each of which is aimed at treating the same disease process and each in a given substandard dosage, it should allow for greater comprehensive efficacy while simultaneously bypassing side-effects, clinically significant medication interactions; and other potentially unforeseen deleterious effects, all because the dosage is small enough and collaborative chemical diversity manifests favorable kinetic dynamics, thereby mitigating unwanted drug effects.

### CROSS-REFERENCE TO RELATED APPLICATION

61499060, Jun. 20, 2011, Cooperative Drug Combination Systems, same inventor's unintentional provisional application abandonment via patent lawyer discrepancy.

### BACKGROUND FOR THE INVENTION

Throughout history, real art is judged upon the ancient Socratic notion of the art of measure, weighing the intellectual good and the bad. In the case of medicine, the pleasure versus pain is expounded upon, from a health perspective, by weighing therapy versus deleterious side-effects. With the current conventional medical model, a single chemical agent is sought, with extensive resources, to counteract a given pathology, with the intent of an all encompassing cure or total symptom alleviation, via one compound that may individually possess multiple mechanisms, but one chemical compound nonetheless. At times, other compounds are added to further the end desired effect of enhancing one ultimate surrogate endpoint. The extensive resources invested in this current single chemical compound model have provided many instances of novel mechanisms to accomplish similar therapeutic goals.

Readily achieving the desired protoplasmic balance may require a revolutionary approach. The therapeutic market is flooded with the current fractionated model of particular mechanisms seemingly not being enhanced to therapeutic potential. With these novel mechanisms of chemical therapy have come novel side-effects, often unwanted. This current gold standard of medication research and development has provided many redundant duplicates, some of which are bereft of creativity and whose existence hinges upon the success of marketing. Consequently, there is a glut of fractionated instances of attempting to treat the totality of a given complex pathological condition, but no standard to capture a greater potential of the currently available technology. The proposed landmark cooperative medication combination therapy may stand as a revolutionary model, a closer attempt to lessen pathology by increasing health and minimizing side-effects, a therapeutic initiative yet to be explored.

It is accepted that most initial teachings regarding abstract concepts and/or concepts with many moving parts need to be simplified. The human body is often simplified for educational reasons. Likewise, medications are simplified for similar inculcation. When the human body is attempted to be understood in relation to medication, then the oversimplifications can become markedly excessive. The lack of fully understanding the relationship of medicine and the human body has been further disconnected through profit marketing, leaving a significant reality gap between the deeply intricate balances within the complexities of human medicine.

It is assumed the history of medicine began when it was noted that people become sick. Currently, conventional medicinal science is a race to uncover the molecular nature of disease, thereby revealing the code to reverse, prevent or lessen disease from such a deciphered grail. It would seem that the single compound treating a corresponding disease was at one point adequate, at least the best offered at the time. Then disease knowledge became more advanced. Simultaneously disease treatment became more complex. Critiques of both became more widespread, some gaining legitimacy. Perhaps the most damaging is that profit has remained profit, but the fetish with such, as noted by Karl Marx, has appeared to only heighten, distorting sensibilities along the way.

Within the social system of medicine, the notoriety, be it corporate or individual, has encouraged a wasteful and disconnected system. Companies are encouraged to keep health information secretive so as to hot impede the ultimate short-term corporate agenda. Researchers are educated in a system that requires such a philosophical doctor to dissect and articulate a reality none have ever considered. These terminal recapitulations isolate and force a sense of disconnect, be it in a cubicle, lab, a hospital bed, or otherwise isolated realm. This paradigm can overlook the obvious or find incentives beyond the common good to disenchant a sustainable idea, an idea guided by a genuine sense of bettering humanity.

Current medical convention views the molecular level of the human body through microscopic receptors. Medications generally bring about a change in the human body through reactions via various receptors. The receptors in turn will generate or disable a series of chemical events in the body. Some medications affect multiple receptors. Modern medicine looks to manipulate receptors so as to best manipulate the human body. New receptors are continually being discovered, as are novel ways to manipulate those receptors. Searching for the latest mechanism manipulation is very much the endeavor for which current research and development resources are geared. The exorbitant hoodwinking involved in clinical trials, evaluating such medication reactions is notable to mention for it is certainly relevant to the advancement of cooperative medication, but beyond the scope of detailing the high level cooperative combination concept.

As it stands, the current model of medically treating a given condition lacks integrity. One way this is well documented is in the 1999 Institute of Medicine research, To Err is Human. From the IOM report, the extensive and costly ramifications of medical errors were exposed with considerable detail. It is noted most medical errors occur at the level of prescribing, often involving dosing errors. Research and development resources have been heavily skewed toward the single compound model eluded herein, and the treatment options for the most studied, most profitable, diseases have consequently become excessive. This has been the status quo, seemingly accepted for decades, as the research and development for the approval of such a given compound is a massive undertaking, making such development feasible only for those wielding tremendous budgets. After further brief oversimplification explanations, it may become clearer why the novel cooperative model presented is revolutionary and will exponentially shift resources toward such a system of development.

Combining medication therapy is not new. It is more of an obvious step in an evolving path to treating a given disease requiring multiple medications. The novelty of the concept being explained reinvents the entire research and development process, current combinations included. Similarly, the combination of medication to best mimic desired human physiology is not new. Oral contraceptives are an example of combinations of medications sought to best mimic the balance of the human body, albeit in a way for which body physiology is tricked. A basic contraceptive combines an estrogen equivalent with a progesterone equivalent. The numbers of combinations using some combination of just one type of estrogen, namely estradiol, are greater than 100. The number of products available in the area of contraception illustrates a great deal of the current research and development model's lack of economic sustainability. The various products differ more in slight nuances of clinical importance; they are less of a pharmacopeia of various chemical mechanisms to prevent pregnancy than they area collection of different chemical structures all of which are designed merely to act upon the very same receptors to prevent ovulation. The different chemical structures tend to act upon the very same receptors. Oral contraceptives are a good example of how the current medication model, including combination therapy, encourages resource ineffectiveness.

As is the case with hormonal therapy, the exceptionally intricate chemical balance of the human body is likely not nearly as well understood as marketing current research would suggest. If one company develops a rudimentary mechanism model, then the competitor will aspire to do the same so as to not lose market share. If the new cooperative model, contained herein, was understood, then that entire process would fluctuate. Most notably, the conventional single chemical model would fluctuate in the direction of what is significantly greater for the patient first, and profit would be a secondary effect.

A fixed dose combination tablet, generally referred to as a polypill, used in the UMPIRE trial aspires some degree of novelty as it sought to achieve better outcomes than its individually prescribed chemical composition. Like the TIPS trials, it combined doses of various mechanisms used to treat cardiovascular disease, included was a 3 mechanism regimen of hypertension medications, with a cholesterol medication and an over-the-counter (OTC) clot prevention medication. It is understood that such a combination is for convenience, increasing me medication regimen adherence of the patient, lending itself to more efficacy with the seemingly consolidated cocktail used for medication administration compliance. Although it is somewhat of a novel idea to combine aspirin and a statin with a few hypertension medication staples, it is much different than using substandard doses to attempt a synergistic effect of consistently lower blood pressure, while decreasing given side-effects. As will be illustrated, the combination of various medications is not new. Combinations are most often done to evade patent expirations, under the guise of ease of patient administration compliance.

The TIPS-2 trial added potassium to the polypill. This has been an addition that could make sense in most potassium depleting diuretic regimens. The addition of potassium would seem to make sense to any clinician familiar with diuretic treatment, but it is just not cost-effective to research and develop such a medication. Diuretics are generally the cheapest medications to use for high blood pressure, and the clinical inertia to use a costly new, albeit more novel potassium containing diuretic would generally not outweigh the cost to use older diuretics that do not contain the potassium replacement.

The novelty of the polypill is subtle, but it takes advantage of the general concept of the compound diseases that compose cardiovascular disease, and instead of using the different tablets for the different diseases encompassing cardiovascular disease, it offers one tablet that would seem to fit well with most patients suffering from cardiovascular disease. The hypertension aspect of cardiovascular disease offered a three mechanism approach to lowering blood pressure. So far, the polypill is the best attempt to advance the soon to be archaic single active medication archetype.

The polypill uses doses that general practitioners typically do not use for newly diagnosed hypertension. This treatment would be more convenient for a given cardiologist seeking to treat a given cardiology patient. As healthcare is further fractionated into specialties, such specialists tend to see general healthcare through a narrower lens, albeit a highly intricate specialized perspective. It would make sense that the more lucrative aspects of healthcare, such as cardiology resources, have progressed the most. However, with all the resources available it would be prudent to begin to shift to the simpler, yet wider scale use of the cooperative medication system model.

The new cooperative model assumes human chemistry is a form of chemical balances. The chemical forces are seen more in light of the extensive chain of events resulting from alterations in the basal human chemical milieu. General concepts of diurnal patterns of chemical activity, such as the role melatonin, growth hormone, and the adrenal gland's adrenaline and cortisol, can each be analyzed to express a lifetime of minutia and subtle intricacies that could still leave many significant questions unanswered. This is just one generally simple concept. There are countless others, especially when pathologic pathways are examined, and the quantity is exponentially greater when juxtaposed with medicinal influences. The wealth of sensory apparatuses, affecting human chemical balance, such as the common experience of mere auditory music, visual aesthetics, or olfactory stimulation bestow alterations in the chemical activity of a human body. Such further abstract phenomena, things not considered ingested, scratches at the human chemical grandeur beyond the scope of either model, but a looming confounding reality nonetheless.

Any given bodily complexity, such as the totality of adrenaline's effects throughout the body, would seem to be understood to a lesser degree than the status quo presumes. Perhaps the not so archaic words of Thomas Edison still hold some truth: Until man duplicates a blade of grass, nature can laugh at his so-called scientific knowledge. Further simplifications of the human body will still help to reveal the underlying complexity and how to best follow advances in understanding. The direct and compensatory mechanisms and such are thought to follow Newtonian laws, in ways which are not always completely understood, to further a chain of chemical events in multiple ways throughout the human body. For example, a given chemical manipulation of cell receptors at a given location, of the human body accompanies a series of events. Some of these events are understood more thoroughly than others. This includes the psychological manipulation of the human mind to alter the intricate chemical balance of the physical human body and vice versa. However, the cooperative model is believed to be more advanced and more physiologically representative than previous disparate corporate interpretations, by taking into account more of that which is readily therapeutically available today, but till now overlooked.

To relate the difference between the current fractionated medical model, it is helpful to consider a given antihypertensive medication. Perhaps the most efficient is a simple diuretic such as hydrochlorothiazide. Diuretics generally promote urine production. They facilitate sodium loss from the plasma and extracellular fluid, via kidney receptors, that brings water and lessens the pressure on the vasculature, as measured with sphygmomanometry. There are various general phases of how they affect blood pressure. There is an initial phase of blood volume loss, but the body learns to compensate for this loss. Eventually diuretics result in vasodilation from an unknown mechanism. Through these mechanisms various hormones are affected. Included in the diuretic effects are alterations in uric acid, glucose, potassium, calcium, sodium, chloride, bicarbonate, hydrogen, etc. The consequent effects of altering these various substances associates diuretics with gouty arthritis, diabetes mellitus, hypokalemia, osteoporosis, dehydration, neurologic disorders, metabolic pH changes, hypotension, etc. Generally diuretics are considered a simple mechanism, altering sodium mechanics in the kidney, and they are deemed clinically safe for the general population. The simple method of increasing urine production does influence various systems in the body; of primary note is a reduction in blood pressure. The other areas affected are not so well studied.

As a government agency regulating the efficacy and safety of prescription medications, the Food and Drug Administration requires the entity applying for approval of a given medication to provide data regarding minimally effective treatment, or starting doses for a given malady for which it has a demonstrative effect. The FDA has standards that can be based on a surrogate endpoint for more easily quantifiable results. For hypertension, blood pressure readings can be established and the medication's resulting reduction compared to placebo is measured and the dose dependent phenomena allows for a minimal dose to be declared effective, sometimes a maximum is determined. This can likewise be done for blood sugar, cholesterol, and standard quality of life surveys, etc, for a given malady studied. The approved starting doses are well documented in readily available tertiary literature, manufacturer package inserts, etc. Doses below the designated effective dose are understood to not be clinically effective, and are consequently not used as novel treatment options. These substandard doses generally do have measurable effects, just not to the degree sought for approval thresholds. Generally substandard doses, as referred in this patent, are those that are lower than the lowest manufactured or approved dose.

Given that doses lower than an FDA designated low dose are not deemed clinically effective, they are not typically manufactured as their use is not a standard of practice. Homeopathy uses substandard doses; however, it does so to such an extreme that the “active” chemical has been subject to such monumental dilution that it has subsequently lost all rational efficacy outside of psychogenic placebo effects. The new model being purported here is wholly distinct from both homeopathy and also “low” combination doses that are noted in some current conventions of therapy. Substandard doses would fall between these two extremes, of homeopathy and already delineated “low” doses, whereby the dose of a single substandard dose has a measurable effect, just not one that meets the FDA cutoff for clinical efficacy when used in isolation (i.e., without additional ingredients used cooperatively to meet a clinical goal). The idea that even minimum low dose prescriptions offer a single chemical potency that significantly alters a fraction of a “known” chemical/mechanical physiologic pathway to such a high degree that the results are deemed clinically significant by the FDA is more associated with the substandard distinction herein.

If a series of substandard dosed cooperative mechanisms can be used to reach one endpoint, such as lower blood pressure, then the various mechanical distinctions can have a synergistically positive effect. In other words, the compensatory mechanisms and time dependent associations with a given chemical can result in a smoother omni-therapeutic result when combined with that which has already been proven to be effective individually.

The inverse would be true for a given side-effect particular for a given mechanism, whereby the substandard combination would result in a diminished incidence of any particular side-effects for a given pharmacologic mechanism. With a lower dose used to accomplish a collaborative endpoint, the resultant individual dose dependent side-effect is proportionally less. In the case of a diuretic, the memetabolic elimination can further effectively reduce the potentially troubling elevations in uric acid, glucose, etc. otherwise found at higher diuretic concentrations.

Perhaps common side-effects among mechanisms will additionally be lessened as the cause of the common side-effect is occurring at a much different rate and time than when occurring with a single mechanism model utilizing higher single-drug potency. Hypotension is a common side-effect among antihypertensives. This side-effect would occur at a lesser rate and consequently to a lesser degree when six mechanisms are only synchronized to lower the blood pressure, not to do it in a collectively organized kinetic time frame beyond the time frame studied and approved. Similarly, there would be a mitigation of rebound hypertension if tapering off the cooperative medication system was desired. Presumably, this too would be the case with a cooperative combination system used to lower blood sugar or symptomatic alleviation with a depression treatment model of this new design.

The novel art described herein may be empirically unproven, but it has the potential to radically change the way medical disease is managed, and in a convenient cost-effective manner. As an unproven concept, without clinical trials, it is described in lengthy terms, often using abstract concepts. The ramifications, both therapeutic and also economic, are difficult to fully quantify. It is anticipated that the invention examples to be provided will help to further illustrate this novel approach with concrete, examples. This paradigm shift could take much of the practice out of medicine, for it utilizes. medications that are already well understood, just uses them more effectively, safer, earlier in the disease process. The medications are even well understood when used together, albeit currently only at individually therapeutic or even a low dose, often when treatment with a given single mechanism has proven inadequate. In some ways this new model for therapeutic treatment is a holistic form of conventional medical therapy, an unequalled fusion of that which is already clinically available, but yet to be tried as detailed. For the sake of pedagogy, practical examples are henceforth provided below.

### BRIEF SUMMARY OF THE INVENTION

The invention advocates the concept of treating patients with beyond lower than manufacturer/government entity determined “low dose” prescription medications, and does so in a cooperative combination of no less than three combined prescription medications. The reason for combining medications is to theoretically achieve more clinically significant results from a variety of substandard proportioned doses of prescription medications. This combination may include the addition of vitamins, minerals, supplements, non-legend medications (OTC), or nutraceuticals. When used individually, the proportionally lower dose of the prescription medication would merely be generally considered clinically insignificant, for the general population, at such a substandard dose. By combining multiple medications, each in their own substandard dosage, it is theorized that the resulting cumulative combination would produce markedly clinically significant efficacy, all while precluding side-effects and interactions for certain indications, the collaboration of which could very well reinvent pharmacological prescribing practices.

### BRIEF DESCRIPTION OF THE SEVERAL VIEWS OF THE CONCEPT

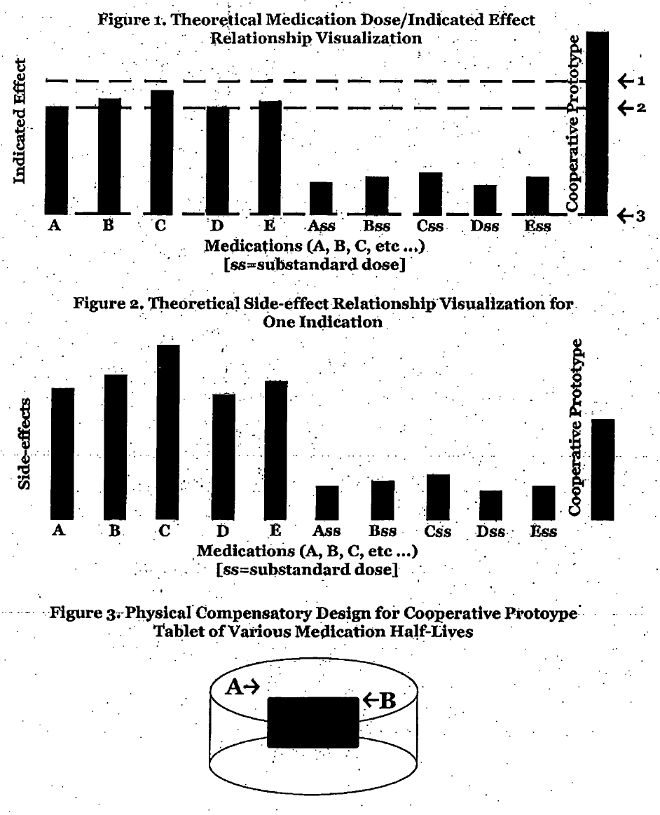

FIG. 1 graphically attempts to quantify and solidify the abstract nature of teaching the details of the cooperative combination medication system. Any of the medication prototypes from Table 1-4 (not to exclude chronic pain management, constipation, etc.) could be applied to the figure, with the conventional low dose medication corresponding to A, B, C, etc. Likewise, the substandard component doses of the cooperative combination medication systems correspond to A (ss), B (ss), C (ss), etc, and are noted to have sub-clinical responses individually. The figure aspires to demonstrate the combination of all of the substandard components would produce a greater indicated effect, as seen with the last graph bar labeled Cooperative Prototype. This represents the untried yet clinically rational concept of cooperative combination medication systems. The dotted line designated as “1” is the indicated effect of that of a low dose medication. This represents the general dose manufactured by a given entity. The dotted line designated as “2” represents the minimum threshold for which the Federal Food and Drug Administration approves a clinically significant indicated effect. Line “3 ” demonstrates the anticipated indicated effects measured by homeopathic doses of medications.

FIG. 2 is similar to FIG. 1, except FIG. 2 compares the same delineated dosages in relation to side-effects. This time the medicinal kinetics illustrate a dynamic that renders the cooperative combination medication system prototype with a comparatively lessened side-effect profile. Likewise, this represents the abstract invention, with many moving clinical parts, proven only with tremendous resources beyond a micro entity's wherewithal.

FIG. 3 offers a medication delivery system that further illustrates the practical application of the invention: “A” is a tablet exterior containing the immediate release long half-life medications, including ¼ of the total dose of short-acting medications, vitamins, minerals, supplements, etc. “B” is a sustained delivery matrix (i.e. potential methylcellulose composite) releasing ¾ of the total short-acting medications over an extended period of time.

TABLE 1
“Syncpress” hypertension prototype tablet teaching example

| Medication | Substandard | | 5 times | | “Low” | | Half- | |
|---|---|---|---|---|---|---|---|---|
| name | Strength (SS) | | SS | | dose/24 hrs | | life | |
| Carvedilol | 1 | mg | 5 | mg | 6.25 | mg | 9 & 7 | h |
| Lisinopril | 2 | mg | 10 | mg | 10 | mg | 12 | h |
| Chlorthalidone | 4 | mg | 20 | mg | 25 | mg | 50 | h |
| Amlodipine | 0.8 | mg | 4 | mg | 2.5 | mg | 40 | h |
| Clonidine | 0.02 | mg | 0.1 | mg | 0.2 | mg | 12 | h |
| Hydralazine | 5 | mg | 25 | mg | 40 | mg | 5 | h |

TABLE 2
“Mellyces” diabetes mellitus prototype tablet teaching example

| Medication | Substandard | | 5 times | | “Low” | | Half- | |
|---|---|---|---|---|---|---|---|---|
| name | Strength (SS) | | SS | | dose/24 hrs | | life | |
| Metformin | 200 | mg | 1000 | mg | 1000 | mg | 6 & 17 | h |
| Sitagliptin | 12.5 | mg | 62.5 | mg | 25 | mg | 12 | h |
| Pioglitazone | 4 | mg | 20 | mg | 15 | mg | 5 & 20 | h |
| Levocarnitine | 200 | mg | 1000 | mg | 990 | mg | — | |
| Glyburide | 0.3 | mg | 1.5 | mg | 2.5 | mg | 10 | h |

TABLE 3
“Eclepid” hyperlipidemia prototype tablet teaching example

| Medication | Substandard | | 5 times | | “Low” | Half- | |
|---|---|---|---|---|---|---|---|
| name | Strength (SS) | | SS | | dose/24 hrs | life | |
| Simvastatin | 4 | mg | 20 | mg | 5 mg | 2 | h |
| Fenofibrate | 5 | mg | 25 | mg | 54 mg | 20 | h |
| Ezetimibe | 0.5 | mg | 2.5 | mg | 10 mg | 22 | h |

TABLE 4
“Lucidotion” depression prototype tablet teaching example

| Medication | Substandard | | 5 times | | “Low” | | Half- | |
|---|---|---|---|---|---|---|---|---|
| name | Strength (SS) | | SS | | dose/24 hrs | | life | |
| Sertraline | 4 | mg | 20 | mg | 25 | mg | 24 | h |
| Venlafaxine | 15 | mg | 75 | mg | 75 | mg | 5 | h |
| Amitriptyline | 4 | mg | 20 | mg | 25 | mg | ~53 | h |
| Mirtazipine | 2 | mg | 10 | mg | 15 | mg | 30 | h |
| Trazodone | 4 | mg | 20 | mg | 25 | mg | 4 & 7 | h |
| Methylphenidate | 0.25 | mg | 1.25 | mg | 2.5 | mg | 3.5 | h |
| Aripiprazole | 0.25 | mg | 1.25 | mg | 2.5 | mg | 75 | h |

List 1a. Available Prescription Hypertension Medications:
Acebutolol—Beta-Blocker (BB)
Aliskiren—Direct Renin Inhibitor
Aliskiren Amiodipine—Combination of Direct Renin Inhibitor/Calcium-Channel Blocker (CCB)
Aliskiren; Amiodipine: Hydrochlorothiazide, HCTZ—Combination of Direct Renin Inhibitor/Calcium-Channel Blocker (CCB)/Thiazide Diuretic
Aliskiren: Hydrochlorothiazide, HCTZ—Combination of Direct Renin Inhibitor/Thiazide Diuretic
Aliskiren: Valsartan—Combination of Calcium-Channel Blocker(CCB)/Angiotensin II Receptor Antagonist
Amiloride—Potassium-Sparing Diuretic
Amiloride: Hydrochlorothiazide, HCTZ—Potassium-Sparing Diuretic/Thiazide Diuretic
Amiodipine—Calcium-Channel Blocker (CCB)
Amiodipine: Atorvastatin—Combination of Calcium-Channel Blocker (CCB)/Statin
Amiodipine: Benazepril—Combination of Calcium-Channel Blocker (CCB)/Angiotensin-Converting Enzyme Inhibitor (ACE-1)
Amiodipine: Hydrochlorothiazide, HCTZ; Olmesartan—Calclum-Channel Blocker (CCB)/Thiazide Diuretic/Angiotensin II Receptor Antagonist (ANG2)
Amlodipine: Hydrochlorothiazide, HCTZ; Valsartan—Combination of Calcium-Channel Blocker (CCB) Thiazide Diuretic/Angiotensin n Receptor Antagonist (ANG2)
Amiodipine: Olmesartan Combination of Calcium-Channel Blocker (CCB)/Angiotensin II Receptor Antagonist (ANG2)
Amiodipine: Telmisartan—Combination of Calcium-Channel Blocker (CCB)/Angiotensin II Receptor Antagonist (ANG2)
Amiodipine: Valsartan—Combination of Calcium-Channel Blocker (CCB)/Angiotensin II Receptor Antagonist (ANG2)
Atenolol—Beta-Blocker (BB)
Atenolol: Chlorthalidone—Combination of Beta-Blocker (BB)/Thiazide Diuretic
Azilsartan—Angiotensin II Receptor Antagonist (ANG2)
Benazepril—Angiotensin-Converting Enzyme Inhibitor (ACE-I)
Benazepril; Hydrochlorothiazide, HCTZ—Combination of Angiotensin II Receptor Antagonist (ANG2)/Thiazide Diuretic
Bendroflumethiazide—Thiazide Diuretic
Bendroflumethiazide; Nadolol—Combination of thiazide Diuretlc/Beta-Blocker (BB)
Betaxolol—Beta-Blocker (BB)
Bisoprolol—Beta-Blocker (BB)
Bisoprolol; Hydrochlorothiazide, HCTZ—Combination of Beta-Blocker (BB)/Thiazide Diuretic
Bumelanide—Loop Diuretic
Candesartan—Angiotensin II Receptor Antagonist (ANG2)
Candesartan; Hydrochlorothiazide, HCTZ—Combination of Angiotensin II Receptor Antagonist (ANG2)/Thiazide Diuretic
Captopril—Angiotension-Converting Enzyme Inhibitor (ACE-I)
Captopril; Hydrochlorothiazide, HCTZ—Combination of Angiotensin-Converting Enzyme Inhibitor (ACE-I)/Thiazide Diuretic
Carteolol—Beta-Blocker (BB)
Carvediiol—Beta-Blocker (BB)
Chlorothiazide—Thiazide Diuretic
Chlorthalidone—Thiazide Diuretic
Chlorthalidone: Clonidine—Combination of Thiazide Diuretic/Central-Acting Adrenergic Agent
Clevidipine—Calcium-Channel Blocker
Clonidine—Central-Acting Adrenergic Agent
Diltiazem—Calcium-Channel Blocker
Diltiazem; Enalapril—Combination of Calcium-Channel Blocker/Angiotensin-Converting Enzyme Inhibitor (ACE-I)
Doxazosin—Alpha-Blocker
Enalapril—Anglotensin-Converting Enzyme Inhibitor (ACE-I)
Enalapril; Felodipine - Combination of Angiotensin-Converting Enzyme Inhibitor (ACE-I)/Calcium-Channel Blocker
Enalapril; Hydrochlorothiazide, HCTZ—Combination of Anglotensin-Converting Enzyme Inhibitor (ACE-I)/Thiazide Diuretic
Eplerenone—Aldosterone Antagonist
Eprosartan—Angiotensin II Receptor Antagonist (ANG2)
Eprosartan; Hydrochlorothiazide, HCTZ—Combination of Angiotensin II Receptor Antagonist (ANG2)/Thiazide Diuretic
Ethacrynic Acid—Loop Diuretic
Felodipine—Calcium-Channel Blocker
Fosinopril—Angiotensin-Converting Enzyme Inhibitor (ACE-I)
Fosinopril; Hydrochlorothiazide, HCTZ—Combination of Angiotensin-Converting Enzyme Inhibitor (ACE-I)/Thiazide Diuretic
Furosemide—Loop Diuretic
Guanabenz—Central-Acting Adrenergic Agent
Guanethidine—Central-Acting Adrenergic Agent
Guanfacine—Central-Acting Adrenergic Agent
Hydralazine—Vasodilator
Hydralazine; Hydrochlorothiazide, HCTZ—Combination of Vasodilator/Thiazide Diuretic
Hydrochlorothiazide; HCTZ—Thiazide Diuretic
Hydrochlorothiazide, HCTZ; Irbesartan—Combination of Thiazide Diuretic/Anglotensin II Receptor Antagonists (ANG2)
Hydrochlorothiazide, HCTZ; Lisinopril—Combination of Thiazide
Hydrochlorothiazide, HCTZ; Losartan—Combination of Thiazide Diuretic/Angiotensin II Receptor Antagonists (ANG2)
Hydrochlorothiazide, HCTZ; Methyldopa—Combination of Thiazide Diuretic/Central-Acting Adrenergic Agent
Hydrochlorothiazide, HCTZ; Metoprolol—Combination of Thiazide Diuretic/Beta-Blocker (BB)
Hydrochlorothiazide, HCTZ; Moexipril—Combination of Thiazide Diuretic/Angiotensin-Converting Enzyme Inhibitor (ACE-I)
Hydrochlorothiazide, HCTZ; Olmesartan—Combination of Thiazide Diuretic/Angiotensin II Receptor Antagonists (ANG2)
Hydrochlorothiazide, HCTZ; Propranolol—Combination of Thiazide Diuretic/Beta-Blocker (BB)
Hydrochlorothiazide, HCTZ; Quinapril—Combination of Thiazide Diuretic/Angiotensin-Converting Enzyme inhibitor (ACE-I)
Hydrochlorothiazide, HCTZ; Spironolactone—Combination of Thiazide Diuretic/Aldosterone Antagonist/Potassium-Sparing Diuretic
Hydrochlorothiazide, HCTZ; Telmisartan—Combination of Thiazide Diuretic/Angiotensin II Receptor Antagonists (ANG2)
Hydrochlorothiazide, HCTZ; Timoiol—Combination of Thiazide Diuretic/Beta-Blocker (BB)
Hydrochlorothiazide, HCTZ; Triamterene—Combination of Thiazide Diuretic/Potassium-Sparing Diuretic
Hydrochlorothiazide, HCTZ; Valsartan—Combination of Thiazide Diuretic/Angiotensin II Receptor Antagonists (ANG2)
Hydroflumethiazide—Thiazide Diuretic
Indapamide—Thiazide Diuretic
Irbesartan—Angiotensin II Receptor Antagonists (ANG2)
Isradipine—Calcium-Channel Blocker
Labetalol—Beta-Blocker (BB)
Lisinopril—Angiotensin-Converting Enzyme Inhibitor (ACE-I)
Losartan—Angiotensin II Receptor Antagonists (ANG2)
Methyclothiazide—Thiazide Diuretic
Methyldopa—Central-Acting Adrenergic Agent
Metolazone—Thiazide Diuretic
Metoprolol—Beta Blocker (BB)
Minoxidil—Vasodilator
Moexipril—Angiotensin-Converting Enzyme Inhibitor (ACR-I)
Nadolol—Beta-Blocker (BB)
Nebiyolol—Beta-Blocker (BB)
Nicardipine—Calcium-Channel Blocker (CCB)
Nifedipine—Calcium-Channel Blocker (CCB)
Nisoldipine—Calcium-Channel Blocker (CCB)
Olmesartan—Angiotensin II Receptor Antagonists (ANG2)
Penbutolol—Beta-Blocker (BB)
Perindopril—Beta-Blocker (BB)
Pindolol—Beta-Blocker (BB)
Prazosin—Alpha-Blocker
Propranolol—Beta-Blocker (BB)
Quinapril—Angiotensin-Converting Enzyme Inhibitor (ACE-I)
Ramipril—Angiotensin-Converting Enzyme Inhibitor (ACE-I)
Reserpine—Alkaloid
Spironolactone—Aldosterone Antagonist/Potassium-Sparing Diuretic
Telmisartan—Angiotensin II Receptor Antagonists (ANG2)
Terazosin—Alpha-Blocker
Timolol—Beta-Blocker (BB)
Torsemide—Loop Diuretic
Trandolapril—Angiotensin-Converting Enzyme Inhibitor (ACE-I)
Trandolapril; Verapamil—Combination of Angiotensin-Converting Enzyme Inhibitor (ACE-I)/Calcium-Channel Blocker (CCB)
Triamterene—Potassium-Sparing Diuretic
Valsartan—Angiotensin II Receptor Antagonists (ANG2)
Verapamil—Calcium-Channel Blocker (CCB)
   List 2a. Available Prescription Diabetes Mellitus Medications:
Acarbose—Alpha-Glucosidase Inhibitor
Acetohexamide—Sulfonylurea
Bromocriptine—Hormone Modifier
Chlorpropamide—Sulfonylurea
Colesovelam—Bile Acid Sequestrant
Exenatide—Incretin Mimetic
Glimepiride—Sulfonylurea
Glimepiride; Pioglitazone—Combination of Sulfonylurea/Thiazolidinedione
Glimepiride: Rosiglitazone—Combination of Sulfonylurea/Thiazolidinedione
Glipizide—Sulfonylurea
Glipizide: Metformin—Combination of Sulfonylurea/Biguanide
Glyburide—Sulfonylurea
Glyburide; Metformin—Combination of Sulfonylurea/Biguanide
Levocarnitine—Glucose tolerance
Linagliptin—Dilpeptidyl Peptidase-4 Inhibitors
Metformin—Biguanide
Metformin; Pioelitazone—Combination of Biguanide/Thiazolidinedione
Metformin; Repaglinide—Combination of Biguanide/Meglitinide
Metformin; Rosiglitazone—Combination of Biguanide/Thiazolidinedione
Metformin: Saxagliptin—Combination of Biguanide/Dipeptidyl Peptidase-4 Inhibitors
Metformin: Sitaeliptin-Combination of Biguanide/Dipeptidyl Peptidase-4 Inhibitors
Miglitol—Alpha-Glucosidase Inhibitors
Nateglinide—Meglitinide
Pioglitazone—Thlazolidinedione
Pramlintide—Amylin analogs
Repaglinide—Meglitinide
Rosiglitazone—Thiazolidinedione
Saxagliptin—Dipeptidyl Peptidase-4 Inhibitors
Sitagliptin—Dipeptidyl Peptidase-4 Inhibitors
Tolazamide—Sulfonylureas
Tolbutamide—Sulfonylureas

### List 3a: Available Prescription Hyperllpidemia Medications:

Amiodipine: Atorvastatin—Combination of Calcium-Channel Blocker (CCB)/HMO-CoA Reductase Inhibitor (Statin)
Aspirin, ASA; Pravastatin—Combination of Salycilate Platelet Inhibitor/HMG-CoA Reductase Inhibitor (Statin).
Atoryastatin—HMG-CoA Reductase inhibitor (Statin)
Cerivastatin—HMG-CoA Reductase Inhibitor (Statin)
Cholestyramine—Bile Acid Sequestrant
Colesevelam—Bile Acid Sequestrant
Colestipol—Bile Acid Sequestrant
Ezetimibe—Cholesterol Absorption Inhibitor
Ezetimibe; Simvastatin—Combination of Cholesterol Absorption Inhibitor/HMG-CoA Reductase Inhibitor (Statin)
Fenofibrate—Fibric Acid Derivatives
Fenofibric Acid—Fibric Acid Derivatives
Fluyastatin—HMG-CoA Reductase Inhibitor (Statin)
Gemfibrozil—Fibric Acid Derivatives
Lovastatin—HMG-CoA Reductase Inhibitor (Statin)
Lovastatin; Niacin—Combination of HMG-CoA Reductase Inhibitor (Stalin)/
Niacin; Simvastatin—Combination of Water-soluble Antilipemic Vitamin/HMG-CoA Reductase Inhibitor (Statin)
Omega-3-acid-ethyl ester—Fat-soluble Antilipemic
Pitavastatin—HMG-CoA Reductase Inhibitor (Statin)
Pravastatin—HMG-CoA Reductase Inhibitor (Statin)
Rosuvastatin—HMG-CoA Reductase Inhibitor (Statin)
Simvastatin—HMG-CoA Reductase Inhibitor (Statin)
   List 4a. Available Depression Symptom Prescription Medications:
Amitriptyline—Tricyclic Antidepressant (TCA)
Amitriptyline: Chlordlazepoxide Combination of Benzodiazepine/Tricyclic Antidepressant (TCA)
Amoxapine—Heterocyclic Antidepressant
Ariplprazole—Atypical Antipsychotic
Bupropion—Multiple Mechanism Antidepressant
Citatopram—Selective Serotonin Reuptake inhibitor (SSRT)
Clomipramine—Tricyclic Antidepressant (TCA)
Desipramine—Tricyclic Antidepressant (TCA)
Desvenlafaxine—Serotonin-Norepinephrlne Reuptake Inhibitor
Doxepin—Tricyclic Antidepressant (TCA)
Duloxetine—Serotonin-Norepinephrine Reuptake Inhibitor
Ephedrine—Adrenergic Agonists
Escitalopram—Selective Serotonin Reuptake inhibitor (SSRI)
Fluoxetine—Selective Serotonin Reuptake inhibitor (SSRT)
Fluoxetine: Olanzapine Combination of Selective Serotonin Reuptake inhibitor (SSRI)/Atypical Antipsychqtic
Fluyoxamine—Selective Serotonin Reuptake Inhibitors (SSRI)
Imlpramine—Tricyclic Antidepressant (TCA)
Isocarboxazid—Monoamine Oxidase Inhibitor (MAOIs)
Maprotiline—Heterocyclic antidepressant
Methylphenidate—Adrenergic Agonist
Mirtazaolne—Heterocyolic Antidepressant
Nefazodone—Phenylpiperazlne Antidepressant
Nortriptyline—Tricyclic Antidepressant (TCA)
Paroxetine—Selective Serotonin Reuptake inhibitor (SSRI)
Phenelzine—Monoamine oxidase inhibitor (MAOIs)
Protriotyline—Tricyclic Antidepressant (TCA)
Quetlapine—Atypical Antipsychotic
Sertraline—Selective Serotonin Reuptake inhibitor (SSRI)
Tranylcypromine—Monoamine oxidase inhibitor (MAOIs)
Trazodone—Heterocyclic antidepressant
Trimipramine—Tricyclic Antidepressant (TCA)
Venlafaxine—Serotonin-Norepinephrlne Reuptake Inhibitors
Vilazodone—Multiple Mechanism Antidepressant
   List 1b. Definitions of Hypertension Mechanisms Used in “Syncpress” Teaching Example:

Beta-Blocker (BB)—this mechanism blocks the effects of the sympathetic nervous system by blocking the effects of neurotransmitters such as norepinephrine. This, blockade will tend to dilate the Vasculature and slow the heart rate. Some beta-blockers, such as carvedilol, also block effects on alpha receptors to further release tension on the vasculature system.

Anglotensin-Convertine Enzyme Inhibitor (ACE-I)—this mechanism involves the prevention of a potent vasoconstrictor called angiotensin 2. There are a number of secondary mechanisms including mild diuresis.

Diuretic—this mechanism hinges upon the removal of sodium from plasma and extracellular fluid volume via the kidneys. The removal of sodium decreases the peripheral vascular resistance

Calcium-Channel Blocker (CCB)—this mechanism prevents the influx of extracellular calcium across the myocardial and vascular cell membranes without changing the plasma levels of calcium. This is another mechanism to decrease tension on the vasculature.

Central-Acting Adrenergic Agent—this mechanism involves agonist effects in the medulla, an effort that reduces the sympathetic response of the body. In simple terms, these medications prevent the release of norepinephrine and can reduce the effects of renin. Both chemicals add to the effects of hypertension.

Vasodilator—some of the total mechanisms are not totally understood, especially as it relates to hydralazine, but this mechanism is know to dilate arterioles more than the venous system when decreasing peripheral vascular resistance.

List 2b. Definitions of Diabetes Mellitus Mechanisms Used in “Mellyces” Teaching Example:

Biguanide—this medication has multiple mechanisms to increase glucose tolerance: decrease glucose production in the liver, decreases the absorption of glucose in the small intestine and increase tissue sensitivity to insulin.

Dipeptidyl Peptidase-4 Inhibitors—this mechanism of glucose control increases insulin synthesis and decreases levels of glucagon, both leading to less sugar in the blood

Sulfonylureas—this mechanism stimulates insulin release from the pancreas to help push sugar into the cells of the body

Thiazolidinedione—this mechanism increases the tissue sensitivity to insulin, including fat tissues, muscle tissues and the liver.

Glucose tolerance—levo carnitine is a B vitamin shown to improve glucose tolerance with a mechanism yet to be elucidated.

List 3b. Definitions of Hyperlipidemia Mechanisms Used in “Eclepid” Teaching Example:

HMG-CoA Reductase Inhibitor (Statin)—this mechanism disrupts an enzyme used to make cholesterol in the liver and it also helps the body to get rid of the worst cholesterol

Fibric Acid Derivatives—this mechanism is not fully understood, but it inhibits the formation of triglycerides and increases the breakdown of certain triglyceride lipoproteins

Cholesterol Absorption Inhibitor—prevents to absorption of cholesterol in the small Intestine

List 4b. Definition of Depression Symptom Alleviation Mechanisms Used in “Lucidotion” Teaching Example:

Selective Serotonin Reuptake inhibitor (SSRI)—this mechanism is not fully understood, but it relates to potent inhibition of serotonin in the central nervous system and potentiating the effects of neurotransmissions associated with pleasure

Serotonin-Norepinephrlne Reuptake Inhibitors—this mechanism relates to both inhibition of serotonin and norepinephrine, with serotonin inhibition often greater. This may also lead to inhibition of dopamine. All of these chemicals are associated with pleasure.

Heterocyclic Antidepressant—this mechanism is not fully understood, but depending on the dose, and even the particular heterocyclic compound, the mechanism relates to serotonin reuptake blocking in the presynaptic membrane. This may involve blocking presynaptic alpha-2 receptors to release serotonin. The release of norepinephrine can occur. Post synaptic serotonin receptors may be blocked and the subtypes of serotonin may differ.

Tricyclic Antidepressant (TCA)—the detailed mechanism is not fully understood, but it is thought that the most important effect is the decreased reuptake of norepinephrine and serotonin but do not effect dopamine reuptake

Adrenergic Agonist—this stimulant mechanism relates to a dopamine uptake blockade of central adrenergic neurons, likely near the brain stem and cerebral cortex. This is associated with pleasurable feelings.

Atypical Antipsychotic—the mechanism relates to manipulation of both dopamine and serotonin receptors, some have enhanced abilities to partially, agonize activity at the D2 receptor since the medication can act as an antagonist at postsynaptic D2 receptors and a weak agonist at presynaptic D2 receptors. This is thought to be the case for aripiprazole.

List 1c. Chemical Structures of Hypertension Medications Used in “Syncpress” Teaching Example:

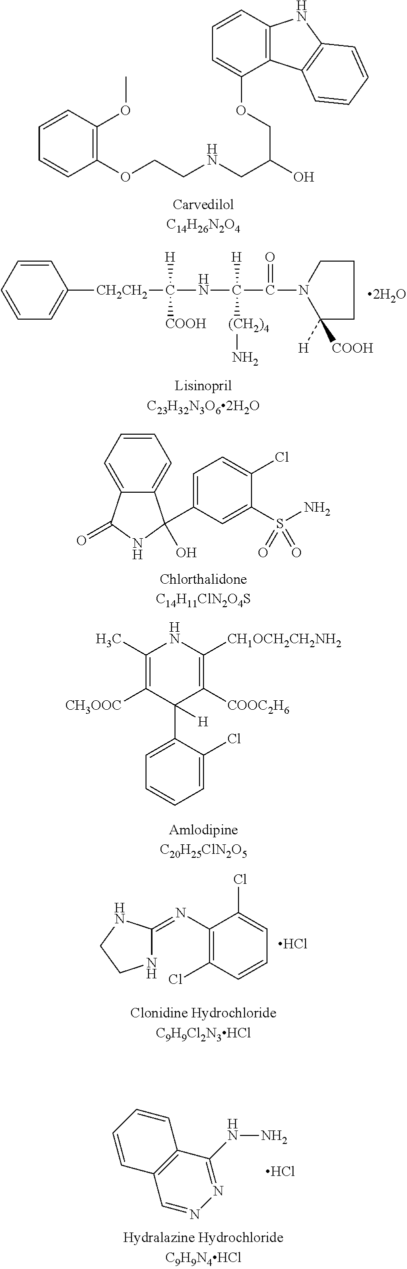

List 2c. Chemical Structures of Diabetes Mellitus Medications Used in “Mellyces” Teaching Example:

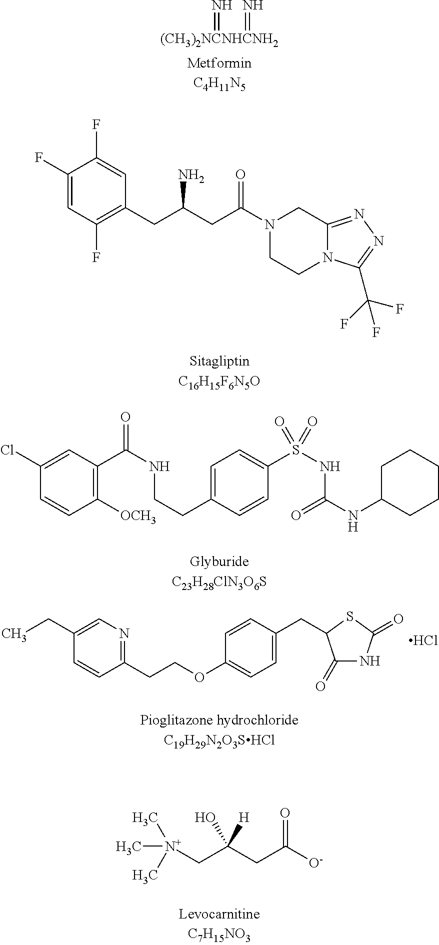

List 3c. Chemical Structures of Hyperlipidemia Medications Used in “Eclepid” Teaching Example:

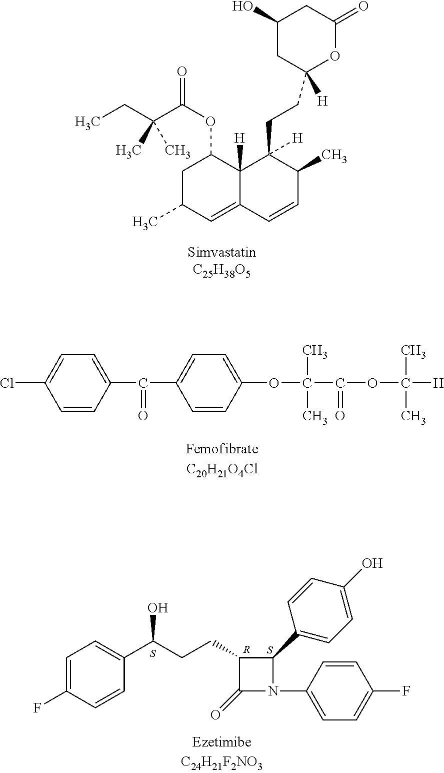

List 4c. Chemical Structures of Depression Medications Used in “Lucidotion” Teaching Example:

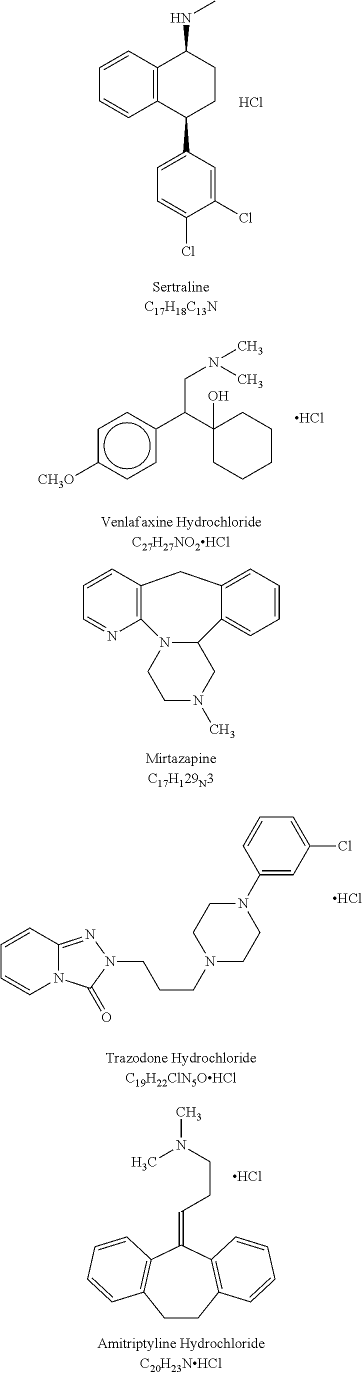

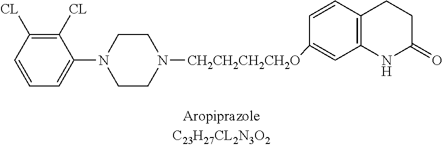

### DETAILED DESCRIPTION OF THE INVENTION

Areas of the greatest concentration of medical resources, such as hypertension, diabetes mellitus, hyperlipidemia and symptomatic treatment of depression can help to best illustrate implementation of the cooperative combination system, from the extensive available research and development in these areas. It would seem the most common diseases of greatest significance in the general population elude even the greatest attempts at controlling their progression. With this in mind, quite often multiple drug regimens are eventually required, but still consequent comorbidities and even direct mortality evade the best attempts with the current treatment approach.

Consider the numerous chemical agents available to treat hypertension. There are slightly more than a half-dozen distinct physiologic mechanisms of action in this pharmacopoeia. Interestingly, there are nearly one hundred available hypertension medications (see List 1a). There are even combination medications, combining various mechanisms. These combinations provide doses that are therapeutic, individually, and are in one tablet/capsule, for advanced hypertension, much like the polypills mentioned earlier. These combinations too are studied and approved to be effective minimally at a given dose. It is crucial to note, whether it is a combination seen on the list provided or the polypill concept, these are not the same as taking six unique, via mechanism of action, medications and decreasing the low dose proportionally, by about ⅙, to a substandard degree, for newly diagnosed uncomplicated hypertension. The combination of 6 chemical mechanisms, contoured to roughly ⅙ of a general low dose is thought to reinvent the current treatment standard. In other words, the doses detailed in this teaching have previously not been considered for use in the medical community, nor, more importantly, has it been postulated for them to be available in combination for commercial use.

However, it is well understood that many diseases, especially chronic ones, benefit from or eventually require more than one agent to adequately mitigate the malady. M R Law and N J Wald, et. al. orchestrated a meta-analysis of 354 randomized trials to appreciate the value of low dose combinations on blood pressure lowering. This low dose corresponds to the FDA designated minimally effective doses. The low dose is less than the dose needed to obtain a typical target blood pressure in a typical hypertensive patient. This analysis was published by the British Medical Journal in 2003. The authors state no trial has studied the effect of three hypertension drugs in combination, but it is suggested the effects would likewise be additive. The authors even report they have a patent application for a formula regarding a combination pill to reduce 4 cardiovascular risk factors. It is assumed the novel concept in the cooperative medication patent application has never occurred to the authors. If such a notion did, then the authors would not limit such a formula to 4 cardiovascular risk factors, certainly not to one given formula. Nothing of the published meta-analysis suggests the authors uncovered the beyond low dose or substandard combination therapy for which no entity has been known to postulate.

The clinical ALLHAT trial illustrates only about one-third of hypertensive patients were treated effectively with a single medication agent. The Seventh Report of the Joint National Committee on Prevention, Detection, Evaluation, and Treatment of High Blood Pressure (JNC 7) states advanced hypertension requires combination therapy. There is much indecision regarding how to standardize the titration of combination therapy and what agents best serve me general population of hypertensive patients. This may well be related to the great concentration of single mechanism medications, used in high concentration, sought to mitigate complex pathologies, especially with the backdrop of significant malpractice litigation when decisions are made with excess ambiguity.

Carvedilol is a hypertension medication that blocks alpha-1 adrenergic receptors, and blocks both beta-1 and beta-2 adrenergic receptors. Lisinopril prevents angiotensin converting enzyme from converting angiotensin 1 to angiotensin 2 to lessen blood pressure. Chlorthalidone prevents reabsorption of sodium and chloride in the kidney, creating a diuretic effect. Amiodipine prevents calcium ion passage into vascular smooth muscle and the myocardium. Clonidine works in the central nervous system to block alpha-2 adrenergic receptors. Hydralazine dilates peripheral vessels directly. Potassium and magnesium are electrolytes lost when using chlorthalidone. Pyridoxine is a vitamin involved in the mechanism of hydralazine. Coenzyme Q10 is found in most cells in the body and its deficiency is associated with many maladies, including hypertension. Under the landmark combination idea, one may take carvedilol 1 mg (low dose=6.25 mg), lisinopril 2 mg (low dose=10 mg), chlorthalidone 4 mg (low dose=25 mg), amiodipine 0.8 mg (low dose=2.5 mg), clonidine 0.02 mg (low dose=0.2 mg), hydralazine 5 mg (low dose=40 mg), potassium 1 meq, pyridoxine 20 mg, magnesium 10 mg and Coenzyme Q10, and combine the substandard doses in a single tablet. This tablet could provide more thorough treatment of the chronic hypertension process (See FIG. 1), and do it in a way that side-effects are mitigated (See FIG. 2).

The idea of allowing the longer half-life medications, such as lismopril and amlodipine to dissolve in an immediate release exterior of the tablet while providing a center matrix for slow-delivery of the remaining anti-hypertensives is a detail beyond the embodiment of this unique idea (See FIG. 3). Either way, the concept lends itself to easier titration and tapering, thereby further lessening side-effects via the various half lives of the various medications. However, a biphasic tablet, possibly scored for further enhanced tittation/tapering efforts, or the need to offer twice daily dosing, will need consideration. Also, determining the practical stability of such a combination of medications is beyond the teaching intent of this novel treatment practice. Further details, such as proportioning this prototype combination from a series of low starting doses or a consensus standard effective treatment dose is left for those with more theoretical medical actuary information, be it for initially treating a disease state of uncomplicated hypertension or any illness sought with this new model.

Likewise, type 2 diabetes mellitus (formerly “non-insulin dependent” or “adult onset” diabetes) is such a wide-scale malady that many medications exist with fewer unique mechanisms. However, a cooperative combination using roughly ¼ the low dose or a consensus standard effective treatment dose could stand to allow the treatment to be effective longer, with greater tolerability, etc. The parts of this potential combination include: metformin, sitagliptin, glyburide: and pioglitazone. Metformin decreases glucose production in the liver. It decreases the absorption of glucose in the small intestine and it increases tissue sensitivity to insulin. Sitagliptin increases insulin synthesis and decreases levels of glucagon. Glyburide stimulates insulin release from the pancreas. Pioglitazone increases insulin sensitivity in the tissues. Levocarnitine is associated with improved glucose utilization. Psylium husk slows the absorption of glucose and aids in the prevention of diabetic gastropathy. Resveratrol is a dietary antioxidant that has been associated with the prevention of heart disease, a pathology associated with diabetes. A metformin 200 mg (low dose=1000 mg), sitagliptin 12.5 mg (low dose=25 mg), glyburide 0.3 mg (low dose=2.5 mg), pioglitazone 4 mg (low dose=15 mg), levocarnitine 200 mg (low dose=990 mg), with psylium husk and resveratrol collaboration could be a revolution in diabetes treatment (See FIGS 1&2).

The idea of allowing the longer half-life medications to dissolve in an immediate release exterior of the tablet and providing a center matrix for slow-delivery of the metformin is a detail beyond the intent of this unique idea (See FIG. 3). Either way, the concept lends itself to easier titration and tapering, thereby further lessening side-effects via the various half lives of the various medications. However, a biphasic tablet, possibly scored for further enhanced titration/tapering efforts, or the need to offer twice daily dosing, will need consideration. The proportions may differ when clinical actuary or trial data is further developed, but the general concept is rational, unique, and untried.

Diabetes Mellitus, like hypertension, is well understood to need combination therapy (See examples in List 2a). It is common consumer knowledge. Consumer Report's Best Buy Drugs, updated December 2012, focused on oral Diabetes medications, clearly stating,/on the recommendation page, that taking more than one diabetes drug is often necessary, but taking more than one diabetes drug raises the risk of adverse effects and increases costs.

Similarly, treating hyperlipidemia could be best accomplished, initially, with a cooperative combination of medications. List 3a includes commercially available combinations that even include medications for different indications, such as hyperlipidemia and hypertension, or hyperlipidemia and clot prevention. As noted, the polypills mentioned previously combine various hypertension medications with an anti-clot mechanism, a hyperlipidemia treatment, some even containing potassium.

Simvastatin reduces 3-hydrpxy-3-methylglutaryl-coenzyme A reductase in such a way that it eliminates much of the fatty substances associated with cholesterol disease. Fenofibrate works in a fashion not fully understood, but inhibits the formation of triglycerides and increases the breakdown of certain triglyceride lipoproteins. Ezetimibe prevents the absorption of cholesterol in the small intestine. Niacin works to decrease bad cholesterol made by the liver, inhibits fat tissue lipolysis, decreased liver esterification, and increases lipoprotein lipase activity. Coenzyme Q10 is found in most cells in the body and. its deficiency is associated with many maladies. The use of a “statin” medication such as simvastatin reduces coenzyme Q10. Omega-3-acid ethyl esters are part of the general population's dietary deficiency and when supplemented are associated with reduced liver triglyceride synthesis. Resveratrol is a dietary antioxidant that has been associated with the prevention of heart disease. The suggestion of combining simvastatin 4 mg (low dose=5 mg), fenofibrate 5 mg (low dose=54 mg), ezetimibe 0.5 mg (only dose=10 mg) with niacin, coenzyme Q10, omega-3-acid ethyl esters, and resveratrol is consistent with the landmark concept of this application. The idea of allowing all but the simvastatin to be released immediately while providing a center matrix for evening delivery of simvastatin is a detail beyond the teaching embodiment of this unique idea (See FIG. 3).

Another readily obvious cooperative combination may even be applied to psychological symptoms. It is often seen in clinical practice, that like the abovementioned combinations, the use of one available prescription medication is too often inadequate to appropriately control a given indication. Often times, this may be related to many complicated confounding factors, but in the case of treating psychological maladies, the placebo effect is much greater. It may be anticipated that a reinvention of the current model can amplify such an effect, but if the chemicals used to symptomatically treat such psychological manifestations offer relief individually, then again the combination would be rational to hold more promise.

Again, this indication offers commercially available combinations (see List 4a). The combinations offered are for comorbitities of depression, and are done so with doses therapeutic for such a comorbity, such as anxiety, bipolar symptoms, or psychosis. Likewise, the anti-depressant is also a therapeutic dose. These combinations are not the cooperative substandard doses combined with at least three mechanisms used for the symptoms of depression treatment.

Sertraline works by selectively inhibiting the reuptake of serotonin, a chemical, process associated with positive feelings. Venlafaxine inhibits the reuptake, of norepinephrine, serotonin, and dopamine, multiple chemicals associated with positive feelings. Mirtazipine effects have not been fully elucidated, but have been associated with antagonizing alpha-2 adrenergic and serotonin 5-HT2 receptors. Similarly, trazodone effects have not been fully elucidated, but have been associated with antagonizing alpha-1 adrenergic and serotonin 5-HT2A and 5-HT2C receptors instead; while also inhibiting the reuptake of serotonin. Likewise, amitriptyline effects are not fully understood, but are associated with the inhibition of the reuptake of norepinephrine and serotonin. The full effects of methylphenidate are not fully known; but it is a central nervous stimulant that affects dopamine transport systems. Ariprazole is also a medication with effects not fully understood, but it is known to partially agonize dopamine and serotonin 5-HT1A receptors while antagonizing serotonin 5-HT2A receptors. The effects of ergocalciferol are extensive but the association of positive feeling and the general dietary deficiency of the population are the aim of its addition. Also, tryptophan is a dietary precursor to serotonin, a key chemical for feelings of. pleasure. Similarly folic acid deficiency is associated with feelings of depression. Sertraline 4 mg (low dose=25 mg), venlafaxine 15 mg (low dose=75 mg), mirtazipine 2 mg (low dose=15 mg), trazodone 4 mg (low dose=25 mg), amitriptyline 4 mg (low dose=25 mg), methylphenidate 0.25 mg (low dose=2.5 mg), aripiprazole 0.25 mg (low dose=2.5 mg), with ergocalcipherol, tryptophan, and folic acid would be as comprehensive a chemical treatment there has ever been for the symptoms of depression, and consequently have the potential to be the safest and most effective at alleviating symptoms (See FIGS. 1 & 2).

The idea of allowing the likely stimulating medications, such as sertraline, methylphenidate, and aripiprazole to dissolve in an immediate release exterior of the tablet, yet contain a center matrix for slow-delivery of the remaining likely sedatives, such as mirtazipine, trazodone and amitriptyline is a detail beyond the intent of teaching this unique idea (See FIG. 3). Either way, the concept lends itself to easier titration and tapering, thereby further lessening side-effects via the various half lives of the various medications. However, a biphasic tablet, possibly scored for further enhanced titration/tapering efforts, or the need to offer twice daily dosing albeit with a non-uniform morning versus evening tablet, will need consideration.

The applications are many, but the above mentioned are just pedagogical examples of this new art applied to help make the abstract art tangible. The prototype tablet proportions have been contoured to not be exact proportions, via the author's experiential knowledge from over a decade of intense study in the given areas of disease treatment. As the potential to better mimic the reverse of a given pathology and consequently lessen side-effects that characterize a given mechanism, the medication interactions should likewise be lessened. Interactions will still be an issue, and in a greater quantity, as they are of clinical concern with the standard single active chemical agent model, but the clinical significance may actually be lessened when multiple mechanisms are represented in the way this art describes. The choice of which medication to prescribe for a particular indication, given hundreds of choices, will be mitigated and radically simplified. Initiating, increasing, or decreasing a given therapeutic regimen will be so rudimentary that it would likely significantly lessen medication errors, namely the Institute of Medicine reported most common errors. In so doing, the comparative effects will be better understood with a larger population being treated similarly. The clinical inertia, such as which single choice is better or which to add to a failing regimen, is drastically reduced & clinical goals may be reached to a tremendous degree.

### REFERENCES

Kohn L T, Corrigan J M, Donaldson M S, editors. To err is human: building a safer health system. A report of the Committee on Quality of Health Care in America, Institute of Medicine. Washington, D.C.: National Academy Press; 2000.
Thorn S, Poulter N, Field J, Patel A, Prabhakaran D, Stanton A, Grobbee D E, Bots M L, Reddy K S, Cidambi R, Bompoint S, Billot L, Rodgers A; UMPIRE Collaborative Group. Effects of a fixed-dose combination strategy on adherence and risk factors in patients with or at high risk of CVD: the UMPIRE randomized clinical trial. JAMA. Sep 4, 2013.
Indian Polycap Study (TIPS); Yusuf S, Pais P, Afzal R, Javier D, Teo K, Eikelboom J, Sigamani A, Mohan V, Gupta R, Thomas N. Effects of a polypill (Polycap) on risk factors in middle-aged individuals without cardiovascular disease (TIPS); a phase II, double-blind, randomised trial. Lancet, Apr. 18, 2009;373(9672):1341-51. Epub 2009 Mar 30.
Comparison of risk factor reduction and tolerability of a full-dose polypill (with potassium) versus low-dose polypill (polycap) in individuals at high risk of cardiovascular diseases: the Second Indian Polycap Study (TIPS-2) investigators. Yusuf S, Pais P, Sigamani A, Xavier D, Afzal R, Gao P, Teo K K. Circ Cardiovasc Qual Outcomes. Jul 1, 2012; 5(4):463-71. Epub 2012 Jul 10.
Law M R, Wald N J, Morris J K, Jordan R E. Value of low dose combination treatment with blood pressure lowering drugs: analysis of 354 randomised trials. BMJ 2003;326:1427-31
The ALLHAT Officers and Coordinators for the: ALLHAT Collaborative Group. Major outcomes in high-risk, hypertensive patients randomized to angiotensin converting enzyme inhibitor or calcium channel blocker vs diuretic. The Antihypertensive and Lipid-Lowering Treatment to Prevent Heart Attack Trial (ALLHAT). JAMA. 2002;288:1981-1997.
Chobanian A V, Bakris G L, Black H R, et al. Seventh Report of the Joint National Committee on Prevention, Detection, Evaluation, and Treatment of High Blood Pressure. Hypertension 2003 ;42;1206-1252.
Consumer Reports Health Best Buy Drugs, “the Oral Diabetes Drugs: Treating Type 2 Diabetes”, Best Buy Drugs (Consumer Reports), retrieved Sep. 18, 2012.
Clinical Pharmacology [database online]. Tampa, Fla.: Gold Standard, Inc.; 2006. URL: http://cp.gsm.com. Updated January 2011.

What is claimed

1. The embodiments of the invention in which an exclusive property or privilege is claimed are defined as follows: I claim the idea of proportionally lower than clinically significant doses, substandard doses, cooperatively combined mostly with multiple prescription medications, differing in mechanism, can safely and effectively treat disease,

2. I claim cooperative medication systems can theoretically be safer and more effective than conventional therapy.

3. I claim the addition of vitamins, minerals, supplements, non-legend medications, and/or nutraceuticals to the cooperative combination medication systems is a novel concept with potential benefits.
